(19) United States
(12) Patent Application Publication
(10) Pub. No.: US 2015/0218090 A1
(43) Pub. Date: Aug. 6, 2015

(54) DOSAGE REGIMEN FOR AN S1P RECEPTOR MODULATOR OR AGONIST

(71) Applicant: NOVARTIS AG, Basel (CH)

(72) Inventor: Erik Wallstroem, Muttenz (CH)

(21) Appl. No.: 14/250,550

(22) PCT Filed: Oct. 18, 2012
(86) PCT No.: PCT/EP2012/070692

§ 371 Date: Apr. 11, 2014

Related U.S. Application Data
(60) Provisional application No. 61/549,847, filed on Oct. 21, 2011.

(51) Int. Cl. C07D 205/04; C07C 251/48

(52) U.S. Cl. CPC C07D 205/04; C07C 251/48; C07C 2101/14

(57) ABSTRACT

This invention relates to a method of treating multiple sclerosis in a mammalian patient, preferably a human, in need of such treatment, comprising administering to said human an S1P receptor modulator or agonist according to a dosing regimen that is determined with reference to the patient's blood lymphocyte count.

### FIELD OF THE INVENTION

The present invention relates to a dosage regimen for a S1P receptor modulator or agonist. More specifically, the present invention relates to a dosage regimen for the treatment of patients suffering from autoimmune diseases or disorders, such as, for example, multiple sclerosis, with a S1P receptor modulator or agonist.

### BACKGROUND OF THE INVENTION

S1P receptor modulators or agonists are compounds which signal as agonists at one or more sphingosine-1 phosphate receptors, for example, S1P1 to S1P8. The binding of an agonist to a S1P receptor may, for example, result in the dissociation of intracellular heterotrimeric G-proteins into Gα-GTP and Gβγ-GTP, and/or increased phosphorylation of the agonist-occupied receptor, and/or the activation of downstream signaling pathways/kinases.

S1P receptor modulators or agonists are useful therapeutic compounds for the treatment of various conditions in mammals, especially in humans. For example, the efficacy of S1P receptor modulators or agonists in the prevention of transplant rejection has been demonstrated in rat (skin, heart, liver, small bowel), dog (kidney), and monkey (kidney) models. In addition, due to their immune-modulating potency, S1P receptor modulators or agonists are also useful for the treatment of inflammatory and autoimmune diseases. The efficacy of the S1P receptor agonist Gilenya® (fingolimod), having the chemical name 2-amino-2-[2-(4-octylphenyl)ethyl]propane-1,3-diol, in the treatment of multiple sclerosis, the chief cause of neurological disability in young adults and the most common demyelinating disorder of the central nervous system, has been demonstrated in humans. Mehling M et al., “FTY720 Therapy Exerts Differential Effects on T Cell Subsets in Multiple Sclerosis”, Neurology, 2008, 71(16): 1261-7; and Kappos L, et al., “Oral Fingolimod (FTY720) for Relapsing Multiple Sclerosis”, New England Journal of Medicine, 2006 Sep. 14; 355(11): 1124-40).

S1P receptor modulators or agonists may produce a negative chronotropic effect at therapeutic doses, i.e., they may reduce the cardiac rhythm, as described, e.g., in Schmouder R. et al., “FTY720: Placebo-Controlled Study of the Effect on Cardiac Rate and Rhythm in Healthy Subjects, J. Clin. Pharmacol. 2006; 46; 895. Administration of 1.25 mg of FTY720 may induce a decrease in heart rate of approximately 8 beats/min. As a consequence of this side effect, the S1P modulator or agonist therapy may need to be initiated under close medical supervision in order to check that the cardiac rhythm is maintained at an acceptable level. This may involve the hospitalization of patients, which makes the treatment more expensive and complicated.

There is a need, with respect to drugs that are SIP receptor modulators or agonists, as there is with drugs in general, to administer the drugs to patients in the lowest efficacious amount, so as to minimize any known side effects of the drug, as well as any unknown side effects. The benefit to a particular patient receiving such a drug would be substantially enhanced if and to the extent that the determination of the lowest efficacious dose is based on properties specific to the individual patient receiving the drug, i.e., if the drug were administered in accordance with an individualized dosing regimen.

The present invention relates to an individualized dosing regimen for the administration of an S1P receptor modulator or agonist for the treatment of an autoimmune disease, e.g., multiple sclerosis.

### SUMMARY OF THE INVENTION

This invention relates to a method of treating an autoimmune disease in a mammalian patient, preferably a human, in need of such treatment, comprising administering to the patient a compound that is an SIP receptor modulator or agonist, or pharmaceutically acceptable salt or prodrug thereof, in accordance with an individualized dosing regimen that is determined with reference to the blood lymphocyte count of the patient. This method is hereinafter referred to as “the Inventive Method”.

A more specific embodiment of this invention relates to the Inventive Method wherein the autoimmune disease is multiple sclerosis.

Another more specific embodiment of this invention relates to the Inventive Method wherein the daily dosage that is determined with reference to the blood lymphocyte count of the patient is adjusted, if necessary, so that it is not less than about 0.5 mg/day and not greater than about 5.0 mg/day.

Another more specific embodiment of this invention relates to the Inventive Method wherein the daily dosage that is determined with reference to the blood lymphocyte count of the patient is adjusted, if necessary, so that it is not less than about 0.2 mg/day and not greater than about 5.0 mg/day.

Another more specific embodiment of this invention relates to the Inventive Method wherein the daily dosage that is determined with reference to the blood lymphocyte count of the patient is adjusted, if necessary, so that it is not less than about 0.25 mg/day and not greater than about 5.0 mg/day.

Another more specific embodiment of this invention relates to the Inventive Method wherein the daily dosage of the S1P receptor modulator or agonist is increased if the patient's blood lymphocyte count is greater than the Blood Lymphocyte Target Level and decreased if the patient's blood lymphocyte count is lower than the Blood Lymphocyte Target Level.

Another more specific embodiment of this invention relates to the Inventive Method wherein the daily dosage of the SIP receptor modulator or agonist is increased if the patient's blood lymphocyte count is greater than the Blood Lymphocyte Target Level and decreased if the patient's blood lymphocyte count is lower than the Blood Lymphocyte Target Level, and wherein the Blood Lymphocyte Target Level is a blood lymphocyte count that is greater than 0.2×10e9/L and less than 0.5×10e9/L.

Another more specific embodiment of this invention relates to the Inventive Method wherein the daily dosage of the S1P receptor modulator or agonist is increased if the patient's blood lymphocyte count is greater than the Blood Lymphocyte Target Level and decreased if the patient's blood lymphocyte count is lower than the Blood Lymphocyte Target Level, and wherein the Blood Lymphocyte Target Level is a blood lymphocyte count that is greater than 0.2×10e9/L and less than 1.0×10e9/L.

Another more specific embodiment of this invention relates to the Inventive Method, wherein the Maintenance Dosage to be administered to a patient is determined by the following process consisting of process steps “a” through “e”:

(a) administering the S1P receptor modulator or agonist, or a pharmaceutically acceptable salt or prodrug thereof, to the patient at an Introductory Dosage that is between about 1.0 mg/day and about 2.0 mg/day, for an Introductory Period;

(b) comparing the blood lymphocyte count of the patient determined at the end of the Introductory Period with the Blood Lymphocyte Target Level;

(c) (i) if the blood lymphocyte count of the patient determined at the end of the Introductory Period is equal to or within the range of the Blood Lymphocyte Target Level, administering the S1P receptor modulator or agonist to the subject at a Maintenance Dosage that is about the same as the Introductory Dosage; or,
(ii) if the blood lymphocyte count of the patient determined at the end of the Introductory Period is lower than the Blood Lymphocyte Target Level, administering the S1P receptor modulator or agonist to the subject for a Post-Introductory Period at a Post-Introductory Dosage that is the greater of (A) between about 40% and about 60% less than the Introductory Dosage and (B) about 0.5 mg/day; or,
(iii) if the blood lymphocyte count of the patient determined at the end of the Introductory Period is greater than the Blood Lymphocyte Target Level, administering the S1P receptor modulator or agonist to the subject for a Post-Introductory Period at a Post-Introductory Dosage that is the lesser of (A) between about 90% and about 110% greater than the Introductory Dosage and (B) about 5.0 mg/day;

(d) if, during the Post-Introductory Period, the patient was administered the S1P receptor modulator or agonist, or a pharmaceutically acceptable salt or prodrug thereof, at a Post-Introductory Dosage that was determined in accordance with step (c) (ii) or step (c) (iii) above, then, comparing the blood lymphocyte count of the patient determined at the end of the Post-Introductory Period with the Blood Lymphocyte Target Level;

(e) (i) if the blood lymphocyte count of the patient determined at the end of the Post-Introductory Period is equal to or within the range of the Blood Lymphocyte Target Level, administering the S1P receptor modulator or agonist to the patient at a Maintenance Dosage that is about the same as the Post-Introductory Dosage; or,
(ii) if the blood lymphocyte count of the patient determined at the end of the Post-Introductory Period is lower than the Blood Lymphocyte Target Level, administering the S1P receptor modulator or agonist to the subject at a Maintenance Dosage that is the greater of (A) between about 40% and about 60% less than the Post-Introductory Dosage and (B) about 0.5 mg/day; or,
(iii) if the blood lymphocyte count of the patient determined at the end of the Post-Introductory Period is greater than the Blood Lymphocyte Target Level, administering the S1P receptor modulator or agonist to the subject at a Maintenance Dosage that is the lesser of (A) between about 90% and about 110% greater than the Post-Introductory Dosage and (B) about 5.0 mg/day.

Another more specific embodiment of this invention relates to the above Inventive Method, comprising steps “a” through “e”, wherein the Introductory Period is about 14 days and wherein the Post-Introductory Period is about 14 days.

Another more specific embodiment of this invention relates to the Inventive Method, wherein the Maintenance Dosage of the S1P receptor modulator or agonist is determined as described in steps “a” through “e” above, and wherein, if the blood lymphocyte count referred to in step “b” is lower than the Blood Lymphocyte Target Level, the S1P receptor modulator or agonist is administered to the subject during the Post-Introductory Period, in accordance with step (c) (ii), at a dosage that is about 50% less than the Introductory Dosage.

Another more specific embodiment of this invention relates to the Inventive Method wherein the Maintenance Dosage of the S1P receptor modulator or agonist is determined as described in steps “a” through “e” above, and wherein, if the blood lymphocyte count referred to in step “b” is greater than the Blood Lymphocyte Target Level, the SIP receptor modulator or agonist is administered to the subject during the Post-Introductory Period, in accordance with step (c) (iii), at a dosage that is about 100% greater than the Introductory Dosage.

Another more specific embodiment of this invention relates to the Inventive Method wherein the Maintenance Dosage of the S1P receptor modulator or agonist is determined as described in steps “a” through “e” above, and wherein the Introductory Dosage is about 1.0 mg/day.

Another more specific embodiment of this invention relates to the Inventive Method wherein the Maintenance Dosage of the S1P receptor modulator or agonist is determined as described in steps “a” through “e” above, and wherein the Introductory Dosage is from about 1.0 mg/day to about 2.0 mg/day.

Another more specific embodiment of this invention relates to the Inventive Method wherein the Maintenance Dosage of the S1P receptor modulator or agonist is determined as described in steps “a” through “e” above, and wherein the Blood Lymphocyte Target Level to which the patient's blood lymphocyte count is compared at the end of the Introductory Period, i.e., as done in step “c” above, is different from the Blood Lymphocyte Target Level to which the patient's blood lymphocyte count is compared at the end of the Post-Introductory Period, i.e., as done in step “e” above.

Another more specific embodiment of this invention relates to the Inventive Method wherein the Maintenance Dosage of the S1P receptor modulator or agonist is determined as described in steps “a” through “e” above, and wherein the Blood Lymphocyte Target Level to which the patient's blood lymphocyte count is compared at the end of the Introductory Period, i.e., as done in step “c” above, is a blood lymphocyte count that is greater than about 0.2×10e9/L and less than about 0.5×10e9/L, and the Blood Lymphocyte Target Level to which the patient's blood lymphocyte count is compared at the end of the Post-Introductory Period, i.e., as done in step “e” above, is a blood lymphocyte count that is greater than about 0.2×10e9/L and less than about 1.0×10e9/L.

Another more specific embodiment of this invention relates to the Inventive Method, wherein the Blood Lymphocyte Target Level used in step (c) and the Blood Lymphocyte Target Level used in step (e) are each, independently, within one of the following ranges:

(A) greater than about 0.2×10e9/L and less than about 1.0×10e9/L;

(B) greater than about 0.2×10e9/L and less than about 0.5×10e9/L;

(C) greater than about 0.2×10e9/L and less than about 0.7×10e9/L;

(D) greater than about 0.3×10e9/L and less than about 0.7×10e9/L; and

(E) greater than about 0.3×10e9/L and less than about 1.0×10e9/L.

Another more specific embodiment of this invention relates to the Inventive Method wherein the S1P receptor modulator or agonist is a compound of Formula Ia or Ib

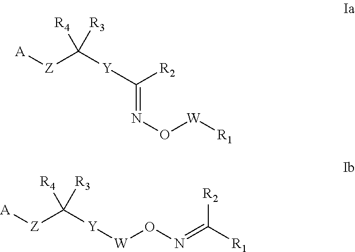

wherein:

A is chosen from —C(O)OR₅, —OP(O)(OR₅)₂, —P(O)(OR₅)₂, —S(O)₂OR₅, —P(O)(R₅)OR₅ and 1H-tetrazol-5-yl; wherein each R₅ is independently chosen from hydrogen and C₁₋₆alkyl;

W is chosen from a bond, C₁₋₃alkylene, C₂₋₃alkenylene;

Y is chosen from C₆₋₁₀aryl and C₂₋₉heteroaryl; wherein any aryl or heteroaryl of Y can be optionally substituted with 1 to 3 radicals chosen from halo, hydroxy, nitro, C₁₋₆alkyl, C₁₋₆alkoxy, halo-substituted C₁₋₆alkyl and halo-substituted C₁₋₆alkoxy;

Z is chosen from:

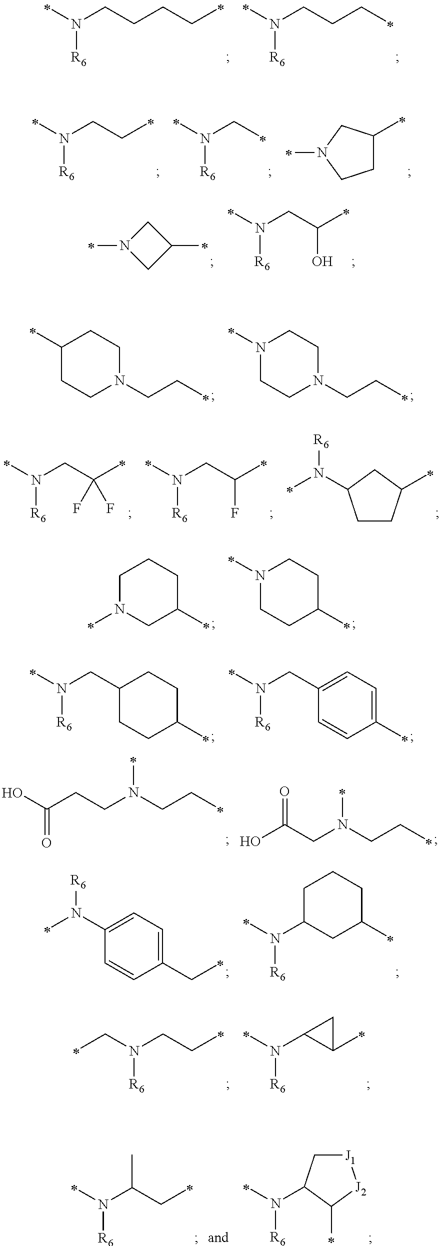

wherein the left and right asterisks of Z indicate the point of attachment between —C(R₃)(R₄)— and A of Formula Ia or Ib, respectively; R₆ is chosen from hydrogen and C₁₋₆alkyl; and J₁ and J₂ are independently methylene or a heteroatom chosen from S, O and NR₅; wherein R₅ is chosen from hydrogen and C₁₋₆alkyl; and any alkylene of Z can be further substituted by one to three radicals chosen from halo, hydroxy, C₁₋₆alkyl; or R₆ can be attached to a carbon atom of Y to form a 5-7 member ring;

R₁ is chosen from C₆₋₁₀aryl and C₂₋₉heteroaryl; wherein any aryl or heteroaryl of R₁ is optionally substituted by a radical chosen from C₆₋₁₀arylC₀₋₄alkyl, C₂₋₉heteroarylC₀₋₄alkyl, C₃₋₈cycloalkylC₀₋₄alkyl, C₃₋₈heterocycloalkylC₀₋₄alkyl or C₁₋₆alkyl; wherein any aryl, heteroaryl, cycloalkyl or heterocycloalkyl group of R₁ can be optionally substituted by one to five radicals chosen from halo, C₁₋₆alkyl, C₁₋₆alkoxy, halo-substituted-C₁₋₆alkyl and halo-substituted-C₁₋₆alkoxy; and any alkyl group of R₁ can optionally have a methylene replaced by an atom or group chosen from —S—, —S(O)—, —S(O)₂—, —NR₅— and —O—; wherein R₅ is chosen from hydrogen or C₁₋₆alkyl;

R₂ is chosen from hydrogen, C₁₋₆alkyl, C₂₋₆alkenyl, C₂₋₆alkynyl and halo substituted C₁₋₆alkyl;

R₃ and R₄ are independently chosen from hydrogen, C₁₋₆alkyl, halo, hydroxy, C₁₋₆alkoxy, halo-substituted C₁₋₆alkyl and halo-substituted C₁₋₆alkoxy; and the N-oxide derivatives, protected derivatives, individual isomers and mixtures of isomers thereof; and the pharmaceutically acceptable salts, solvates (e.g., hydrates) and prodrugs of such compounds.

The above compounds of formula Ia and Ib may contain chiral centers and therefore may exist in different enantiomeric and diastereomeric forms. Individual isomers can be obtained by known methods, such as optical resolution, optically selective reaction, or chromatographic separation in the preparation of the final product or an intermediate. Formulae Ia and Ib above include all optical isomers and all stereoisomers of compounds of the formula I, both as racemic mixtures and as individual enantiomers and diastereoismers of such compounds, and mixtures thereof.

Formulae Ia and Ib above also include isotopically labelled compounds, which are identical to those recited in formulae Ia and Ib, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹O, ¹⁴O, ¹⁵ N, ¹⁸O, ¹⁷O, ³¹P, ³²P, ³⁵S, ¹⁸F, and ³⁶Cl, respectively.

In so far as the compounds of formulae Ia and Ib may have acidic or basic moieties, they are capable of existing in the free acid or free base form and are also capable of forming a wide variety of different salts with various inorganic and organic acids or bases. Although such salts must be pharmaceutically acceptable for administration to animals, it is often desirable in practice to initially isolate the free base or free acid from the reaction mixture as a pharmaceutically unacceptable salt and then convert it to the free base or free acid by treatment with an alkaline or acidic reagent, and thereafter convert the free acid or base to a pharmaceutically acceptable acid or base addition salt. The acids that are used to prepare the pharmaceutically acceptable acid addition salts of the aforementioned base compounds Formula Ia and Ib are those which form non-toxic acid addition salts, e.g., salts containing pharmaceutically acceptable anions, such as the hydrochloride, hydrobromide, hydroiodide, nitrate, sulfate or bisulfate, phosphate or acid phosphate, acetate, lactate, citrate or acid citrate, tartrate or bi-tartrate, succinate, maleate, fumarate, hemi-fumarate, gluconate, saccharate, benzoate, methanesulfonate, ethanesulfonate, benzenesulfonate, p-toluenesulfonate and pamoate (e.g., 1,1′-methylene-bis-(2-hydroxy-3-naphthoate)) salts. The bases that are used to prepare the pharmaceutically acceptable base addition salts of the aforementioned base compounds of Formula Ia and Ib are those which form non-toxic acid addition salts, e.g., salts of metals such as sodium, Potassium, calcium and aluminium, salts with amines, such as triethylamine and salts with dibasic amino acids, such as lysine.

Another more specific embodiment of this invention relates to the Inventive Method wherein the S1P receptor modulator or agonist is 1-{4-[1-(4-cyclohexyl-3-trifluoromethyl-benzyloxyimino)-ethyl]-2-ethyl-benzyl}-azetidine-3-carboxylic acid, or a pharmaceutically acceptable salt, solvate or prodrug thereof.

Another more specific embodiment of this invention relates to the Inventive Method wherein the S1P receptor modulator or agonist is the hemifumarate salt of 1-{4-[1-(4-cyclohexyl-3-trifluoromethyl-benzyloxyimino)-ethyl]-2-ethyl-benzyl}-azetidine-3-carboxylic acid.

This invention also relates to a kit containing daily units of medication of an SIP receptor modulator or agonist, or a pharmaceutically acceptable salt or prodrug thereof, for the treatment of multiple sclerosis in a mammalian patient, preferably a human, in need of such treatment, wherein the daily dosage of the S1P receptor modulator or agonist, or a pharmaceutically acceptable salt, solvate or prodrug thereof, is determined with reference to the blood lymphocyte count of the patient.

A more specific embodiment of this invention relates to a kit, as described above, wherein the daily dosage of the S1P receptor modulator or agonist, or a pharmaceutically acceptable salt or prodrug thereof, is the Maintenance Dosage that was determined using the following method:

(a) administering the S1P receptor modulator or agonist, or a pharmaceutically acceptable salt or prodrug thereof, to the patient at an Introductory Dosage that is between about 1.0 mg/day and about 2.0 mg/day, for an Introductory Period;

(b) comparing the blood lymphocyte count of the patient determined at the end of the Introductory Period with the Blood Lymphocyte Target Level;

(c) (i) if the blood lymphocyte count of the patient determined at the end of the Introductory Period is equal to or within the range of the Blood Lymphocyte Target Level, administering the S1P receptor modulator or agonist to the subject at a Maintenance Dosage that is about the same as the Introductory Dosage; or,
(ii) if the blood lymphocyte count of the patient determined at the end of the Introductory Period is lower than the Blood Lymphocyte Target Level, administering the S1P receptor modulator or agonist to the subject for a Post-Introductory Period at a Post-Introductory Dosage that is the greater of (A) between about 40% and about 60% less than the Introductory Dosage and (B) about 0.5 mg/day; or,
(iii) if the blood lymphocyte count of the patient determined at the end of the Introductory Period is greater than the Blood Lymphocyte Target Level, administering the S1P receptor modulator or agonist to the subject for a Post-Introductory Period at a Post-Introductory Dosage that is the lesser of (A) between about 90% and about 110% greater than the Introductory Dosage and (B) about 5.0 mg/day;

(d) if, during the Post-Introductory Period, the patient was administered the S1P receptor modulator or agonist, or a pharmaceutically acceptable salt or prodrug thereof, at a Post-Introductory Dosage that was determined in accordance with step (c) (ii) or step (c) (iii) above, then, comparing the blood lymphocyte count of the patient determined at the end of the Post-Introductory Period with the Blood Lymphocyte Target Level;

(e) (i) if the blood lymphocyte count of the patient determined at the end of the Post-Introductory Period is equal to or Within the range of the Blood Lymphocyte Target Level, administering the S1P receptor modulator or agonist to the patient at a Maintenance Dosage that is about the same as the Post-Introductory Dosage; or,
(ii) if the blood lymphocyte count of the patient determined at the end of the Post-Introductory Period is lower than the Blood Lymphocyte Target Level, administering the S1P receptor modulator or agonist to the subject at a Maintenance Dosage that is the greater of (A) between about 40% and about 60% less than the Post-Introductory Dosage and (B) about 0.5 mg/day; or,
(iii) if the blood lymphocyte count of the patient determined at the end of the Post-Introductory Period is greater than the Blood Lymphocyte Target Level, administering the S1P receptor modulator or agonist to the subject at a Maintenance Dosage that is the lesser of (A) between about 90% and about 110% greater than the Post-Introductory Dosage and (B) about 5.0 mg/day.

Another more specific embodiment of this invention is a kit, as described above, wherein the SIP receptor modulator or agonist is 1-{4-[1-(4-cyclohexyl-3-trifluoromethyl-benzyloxyimino)-ethyl]-2-ethyl-benzyl}-azetidine-3-carboxylic acid, or a pharmaceutically acceptable salt or prodrug thereof.

Another more specific embodiment of this invention relates to a kit, as described above, wherein the SIP receptor modulator or agonist is the hemifumarate salt of 1-{4-[1-(4-cyclohexyl-3-trifluoromethyl-benzyloxyimino)-ethyl]-2-ethyl-benzyl}-azetidine-3-carboxylic acid.

Another more specific embodiment of this invention relates to a kit, as described above, wherein the daily dosage of the S1P receptor modulator or agonist, or a pharmaceutically acceptable salt or prodrug thereof, is the Maintenance Dosage determined using steps “a” to “e” above” and wherein the Introductory Period referred to in steps “a”, “b” and “c” is about 14 days and wherein the Post-Introductory Period referred to in steps “c”, “d” and “e” is about 14 days.

Another more specific embodiment of this invention relates to a kit, as described above, wherein the Maintenance Dosage of the S1P receptor modulator or agonist is determined as described in steps “a” through “e” above, and wherein, if the blood lymphocyte count referred to in step “b” is lower than the Blood Lymphocyte Target Level, the S1P receptor modulator or agonist is administered to the subject during the Post-Introductory Period, in accordance with step (c) (ii), at a dosage that is about 50% less than the Introductory Dosage.

Another more specific embodiment of this invention relates to a kit, as described above, wherein the Maintenance Dosage of the S1P receptor modulator or agonist is determined as described in steps “a” through “e” above, and wherein, if the blood lymphocyte count referred to in step “b” is greater than the Blood Lymphocyte Target Level, the S1P receptor modulator or agonist is administered to the subject during the Post-Introductory Period, in accordance with step (c) (iii), at a dosage that is about 100% greater than the Introductory Dosage.

Another more specific embodiment of this invention relates to a kit, as described above, wherein the Maintenance Dosage of the S1P receptor modulator or agonist is determined as described in steps “a” through “e” above, and wherein the Introductory Dosage is about 1.0 mg/day.

Another more specific embodiment of this invention relates to a kit, as described above, wherein the Maintenance Dosage of the S1P receptor modulator or agonist is determined as described in steps “a” through “e” above, and wherein the Blood Lymphocyte Target Level to which the patient's blood lymphocyte count is compared at the end of the Introductory Period, i.e., as specified in step “c” above, is different from the Blood Lymphocyte Target Level to which the patient's blood lymphocyte count is compared at the end of the Post-Introductory Period, i.e., as specified in step “e” above.

Another more specific embodiment of this invention relates to a kit, as described above, wherein the Blood Lymphocyte Target Level used in step (c) and the Blood Lymphocyte Target Level used in step (e) are each, independently, within one of the following ranges:

(A) greater than about 0.2×10e9/L and less than about 1.0×10e9/L;

(B) greater than about 0.2×10e9/L and less than about 0.5×10e9/L;

(C) greater than about 0.2×10e9/L and less than about 0.7×10e9/L;

(D) greater than about 0.3×10e9/L and less than about 0.7×10e9/L; and

(E) greater than about 0.3×10e9/L and less than about 1.0×10e9/L.

Another more specific embodiment of this invention relates to a kit, as described above, wherein the Maintenance Dosage of the S1P receptor modulator or agonist is determined as described in steps “a” through “e” above, and wherein the Blood Lymphocyte Target Level to which the patient's blood lymphocyte count is compared at the end of the Introductory Period, i.e., as specified in step “c” above, is a blood lymphocyte count that is greater than 0.2×10e9/L and less than 0.5×10e9/L, and the Blood Lymphocyte Target Level to which the patient's blood lymphocyte count is compared at the end of the Post-Introductory Period, i.e., as specified in step “e” above, is a blood lymphocyte count that is greater than 0.2×10e9/L and less than 1.0×10e9/L.

This invention also relates to the use of a S1P receptor modulator or agonist in the manufacture of a medication for the treatment of an autoimmune disease in a mammalian, including a human, patient, wherein said medication is administered to the patient in accordance with a dosing regimen that is determined with reference to the blood lymphocyte count of the patient.

A more specific embodiment of this invention relates to the use of a S1P receptor modulator or agonist in the manufacture of a medication for the treatment of an autoimmune disease in a mammalian, including a human, patient, wherein said medication is administered to the patient in accordance with a dosing regimen that is determined with reference to the blood lymphocyte count of the patient, and wherein the S1P receptor modulator or agonist is the hemifumarate salt of 1-{4-[1-(4-cyclohexyl-3-trifluoromethyl-benzyloxyimino)-ethyl]-2-ethyl-benzyl}-azetidine-3-carboxylic acid, and wherein the Maintenance Dosage to be administered to the patient is determined using the process of steps “a” through “e” above.

Other S1P receptor modulators or agonists that can be used in the Inventive Method and the more specific embodiments of this invention that are not restricted to the use of specific S1P receptor modulators or agonists include, but are not limited to, those referred to in the following references, which are incorporated herein by reference in their entirety: WO 04/103306, WO 05/000833, and WO 05/103309.

Preferred S1P receptor modulators or agonists that can be used in the methods and kits of this invention include, for example, compounds which, in addition to their SIP binding properties, also have accelerating lymphocyte homing properties. For example, such compounds may elicit lymphopenia resulting from a re-distribution of lymphocytes from the circulation to the secondary lymphatic tissue, which is preferably reversible, without evoking a generalized immunosuppression. Suitably, naïve cells are sequestered and CD4/CD8 T-cells and B-cells from the blood are stimulated to migrate into lymph nodes (LN) and Peyer's patches (PP).

Further aspects and embodiments are provided in the Detailed Description Of the Invention.

### DETAILED DESCRIPTION OF THE INVENTION

The present inventors have surprisingly found that the blood lymphocyte count of a patient in need of treatment with an S1P receptor modulator or agonist for the treatment of multiple sclerosis is related to status or progression of the disease, and, in particular, to the number of lesions observed in an MRI scan, and that this relationship can be utilized to improve the treatment benefit-risk ratio for such patients. Individualizing the dosing regimen of these drugs will likely result in a reduction of individual variability in responses to treatment among patients while improving the safety profile of the drug through use of the lowest effective dose.

The following definitions define the meanings of certain terms, as they are used in this application.

The term “alkyl”, as used herein, unless otherwise indicated, includes saturated monovalent hydrocarbon radicals having straight, branched or cyclic moieties or combinations thereof. Examples of “alkyl” groups include, but are not limited to, methyl, ethyl, propyl, isopropyl, butyl, iso- sec- and tert-butyl, pentyl, hexyl, heptyl, 3-ethylbutyl, cyclopropyl, cyclobutyl, cyclopentyl, cyclohexyl, cycloheptyl, norbornyl, and the like.

The term “alkoxy”, as used herein, unless otherwise indicated, means “alkyl-O-”, wherein “alkyl” is as defined above. Examples of “alkoxy” groups include, but are not limited to, methoxy, ethoxy, propoxy, butoxy and pentoxy.

The term “alkenyl”, as used herein, unless otherwise indicated, includes unsaturated hydrocarbon radicals having one or more double bonds connecting two carbon atoms, wherein said hydrocarbon radical may have straight, branched or cyclic moieties or combinations thereof. Examples of “alkenyl” groups include, but are not limited to, ethenyl, propenyl, butenyl, pentenyl, and dimethylpentyl, and include E and Z forms where applicable.

The term “alkynyl”, as used herein, unless otherwise indicated, includes unsaturated hydrocarbon radicals having one or more triple bonds connecting two carbon atoms, wherein said hydrocarbon radical may have straight, branched or cyclic moieties or combinations thereof. Examples of “alkynyl” groups include, but are not limited to, ethynyl, propynyl, butynyl, pentynyl, and dimethylpentynyl.

The term “aryl”, as used herein, unless otherwise indicated, includes an aromatic ring system with no heteroatoms, e.g., phenyl or naphthyl, which can be either unsubstituted or substituted with one, two or three substituents selected from the group consisting of halo, (C₁-C₄)alkyl optionally substituted with from one to three fluorine atoms and (C₁-C₄)alkoxy optionally substituted with from one to three fluorine atoms.

The term “cycloalkyl”, as used herein means a saturated, cyclic hydrocarbon moiety.

The term “heteroaryl”, as used herein, unless otherwise indicated, includes an aromatic heterocycle containing five or six ring members, of which from 1 to 4 can be heteroatoms selected, independently, from N, S and O, and which rings can be unsubstituted, monosubstituted or disubstituted with substituents selected, independently, from the group consisting of halo, (C₁-C₄)alkyl, and (C₁-C₄)alkoxy, optionally substituted with from one to three fluorine atoms;

The term “heterocycloalkyl”, as used herein, unless otherwise indicated, means a cycloalkyl moiety wherein one or more of the carbon atoms are replaced with a heteroatom selected independently, from N, S and O.

The term “heterocyclic”, as used herein, means a 5- to 7 membered heterocyclic group having 1 to 3 heteroatoms selected from S, O and N. Examples of such heterocyclic groups include the heteroaryl groups indicated above, and heterocyclic compounds corresponding to partially or completely hydrogenated heteroaryl groups, e.g., furyl, thienyl, pyrrolyl, azepinyl, pyrazolyl, imidazolyl, oxazolyl, isoxazolyl, thiazolyl, isothiazolyl, 1,2,3-oxadiazolyl, triazolyl, tetrazolyl, thiadiazolyl, pyranyl, pyridyl, pyridazinyl, pyrimidinyl, pyrazinyl, tetrahydropyranyl, morpholinyl, thiomorpholinyl, pyrrolidinyl, pyrrolyl, imidazolidinyl, pyrazolidinyl, piperidinyl, piperazinyl, oxazolidinyl, isoxazolidinyl, thiazolidinyl or pyrazolidinyl. Preferred heterocyclic groups are 5- or 6-membered heteroaryl groups and the most preferred heteocyclic group is a morpholinyl, thiomorpholinyl or piperidinyl group.

The terms “halo” and “halogen”, as used herein, unless otherwise indicated, include, fluoro, chloro, bromo and iodo.

The term “methylene”, as used herein, means —CH₂—.

The term “alkylene”, as used herein, means —(CH₂)ₙ— wherein n is greater than or equal to 1.

The term “alkenylene”, as used herein, means an alkylene group of at least two carbon atoms, wherein one or more pairs of adjacent carbon atoms are double bonded to each other and not double bonded to another carbon atom.

The term “prodrug”, as used herein, means a substance that is administered to a patient in an inactive or significantly less active form, which is converted, via metabolism in vivo, into an active or significantly more active form. Prodrugs include a functional group is reversible derivative of a functional group of the corresponding drug. Typically, such prodrugs are transformed to the active drug by hydrolysis. Examples are the following:

Functional Group Reversible Derivative
Carboxylic acid Esters, including e.g. alkyl and acyloxyalkyl esters; amides
Alcohol Esters, including e.g. sulfates and phosphates as well as carboxylic acid (e.g. alkanoic acid) esters
Amine Amides, carbamates, imines, enamines,
Carbonyl (aldehyde, Imines, oximes, acetals/ketals, enol esters, ketone) oxazolidines and thiazoxolidines

Prodrugs also include compounds that can be converted into the active drug by an oxidative or reductive reaction. Examples of oxidation activation reactions that convert prodrugs into their corresponding active drugs are N- and O-dealkylation, oxidative deamination, N-oxidation, and epoxidation reactions. Examples of reductive activation reactions that convert prodrugs into their corresponding active drugs are azo reduction, disulfide reduction, bioreductive alkylation and nitro reduction reactions. Also included as metabolic activations of prodrugs are nucleotide activation, phosphorylation activation and decarboxylation activation reactions. For additional information, see “The Organic Chemistry of Drug Design and Drug Action”, R B Silverman (particularly Chapter 8, pages 497 to 546), incorporated herein by reference.

The term “treating”, as used herein, refers to reversing, alleviating, inhibiting the progress of, or preventing the disorder or condition to which such term applies, or preventing one or more symptoms of such condition or disorder. The term “treatment”, as used herein, refers to the act of treating, as “treating” is defined immediately above.

The term “Blood Lymohocyte Target Level”, as used herein, means the blood lymphocyte count, or range of blood lymphocyte counts, that is desired as being associated with the desired efficacy of the S1P receptor modulator or agonist.

The term “Introductory Period”, as used herein, means the period throughout which a patient is dosed with an S1P receptor modulator or agonist at the Introductory Dosage, and ending at a time, after lymphocyte blood levels have had time to become stabilized, when the patient's blood lymphocyte count is first compared to the Blood Lymphocyte Target Level to determine whether a change in dosage is warranted. The Introductory Period need not begin with the initial dosing of a patient with an S1P receptor modulator or agonist. For example, the initial dose of an S1P receptor modulator or agonist given to a patient may be very low, e.g., about 0.25 mg, and followed by slight increases in dosing daily as part of a titration regimen aimed at ameliorating the decrease in heart rate sometimes associated with S1P receptor modulators or agonists. Such a regimen does not define the Introductory Period. During the Introductory Period associated with this invention, the patient is being administered only one dosage and during this period the lymphocyte blood levels have time to become stabilized.

The term “Introductory Dosage”, as used herein, means the initial dosage of the S1P receptor modulator or agonist that is administered to a patient as part of the individualized dosing regimen that is determined with reference to the blood lymphocyte count of the patient, i.e., as part of the Inventive Method. It is possible that a patient designated to receive individualized dosing of an S1P receptor modulator or agonist in accordance with the individualized dosing method of this invention, i.e., the Inventive Method, is already being administered an S1P receptor modulator or agonist in accordance with another dosing regimen, such that the daily dosage that the patient is receiving is less than about 1.0 mg/day or greater than about 2.0 mg/day. For such a patient, the Introductory Dosage would be the initial dosage to which the dosage the patient was previously receiving is adjusted to begin the Inventive Method.

The term “Post-Introductory Dosage”, as used herein, means the dosage of an S1P receptor modulator or agonist that is administered to a patient, in accordance with steps “a” through “e” above of the individualized dosing method of this invention, during the Post-Introductory Period.

The term “Post-Introductory Period”, as used herein, means the period beginning with the first dosing of a patient, after the Introductory Period, with an S1P receptor modulator or agonist using a dosage that was determined based on the first comparison of the patient's blood lymphocyte count with the Blood Lymphocyte Target Level for the purpose of determining whether a dose adjustment is advisable, and ending with the first comparison thereafter of the patient's blood lymphocyte count with the Blood Lymphocyte Target Level for the purpose of determining whether a dose adjustment is advisable.

The term “Maintenance Dosage”, as used herein, means the dosage that is determined, in accordance with steps “a” through “e” above of the individualized dosing method of this invention, to be the dosage that should be maintained for a particular patient. In accordance with steps “a” through “e” above, the Maintenance Dosage will be either: (A) the dosage of the S1P receptor modulator or agonist that is administered to a patient during a period long enough for the lymphocyte count to stabilize and after which it is determined that the blood lymphocyte count of the patient is equal to or within the range of the Blood Lymphocyte Target Level; or (B) about 0.5 mg/day; or (C) about 4.0 mg/day. The Maintenance Dosage is, preferably, the lowest therapeutically effective dosage, i.e., the lowest dosage of the S1P receptor modulator or agonist necessary to effectively treat the intended disease or condition (i.e., so that the subject shows reduced signs or symptoms of the disease to be treated or prevented, or, preferably, no signs and symptoms of the disease).

Examples of specific compounds of the Formulae Ia and Ib that are useful for the purposes of the present invention include:

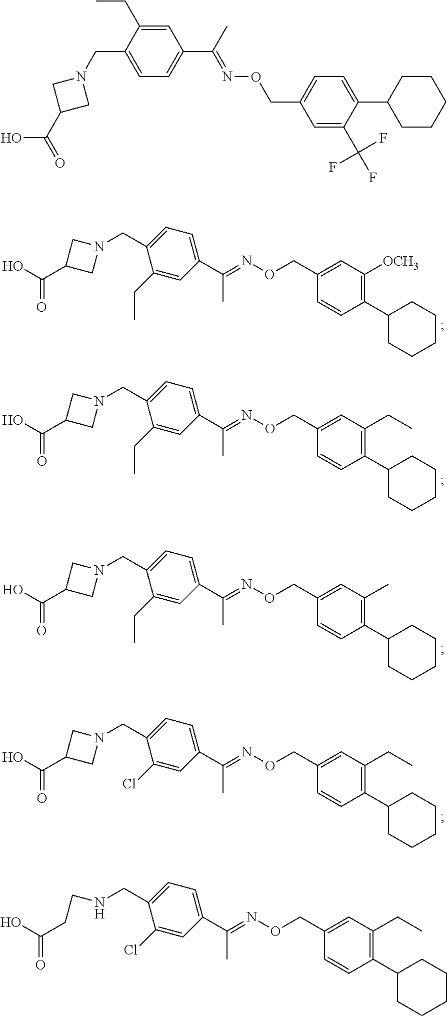 [SMILES: CCc1cc(/C(C)=N/OCc2ccc(C3CCCCC3)c(C(F)(F)F)c2)ccc1CN1CC(C(=O)O)C1 ; CCc1cc(/C(C)=N/OCc2ccc(C3CCCCC3)c(OC)c2)ccc1CN1CC(C(=O)O)C1 ; CCc1cc(/C(C)=N/OCc2ccc(C3CCCCC3)c(CC)c2)ccc1CN1CC(C(=O)O)C1 ; CCc1cc(/C(C)=N/OCc2ccc(C3CCCCC3)c(C)c2)ccc1CN1CC(C(=O)O)C1 ; CCc1cc(CO/N=C(\C)c2ccc(CN3CC(C(=O)O)C3)c(Cl)c2)ccc1C1CCCCC1 ; CCc1cc(CO/N=C(\C)c2ccc(CNCCC(=O)O)c(Cl)c2)ccc1C1CCCCC1]

and the pharmaceutically acceptable salts, solvates and prodrugs thereof.

Other examples of compounds of the formulae Ia and Ib that can be employed in the methods and kits of the present invention are the compounds of the Examples set forth in Table 1 of WO 2004/103306, the content of which is hereby incorporated herein by reference. Such compounds can be prepared using the synthetic methods described in this reference.

The S1P receptor agonist or modulators employed in the methods and kits of this invention may be selective for the S1P₁ receptor. For example, a compound which possesses selectivity for the S1P₁ receptor over the S1P₃ receptor of at least 20 fold, e.g., 100, 500, 1000 or 2000 fold, as measured by the ratio of EC₅₀ for the S1P₁ receptor to the EC₅₀ for the S1P₃ receptor as determined using a ³⁵S-GTPγS binding assay, and wherein said compound has an EC₅₀ for binding to the S1P1 receptor of 100 nM or less as evaluated by the ³⁵S-GTPγS binding assay.

The ³⁵S-GTPγS binding assay is described in WO03/097028 and DS. Im et al., Mol. Pharmacol. 2000; 57: 753. Briefly, ligand-mediated GTPγS binding to G-proteins is measured in GTP binding buffer (in mM: 50 HEPES, 100 NaCl, 10 MgCl₂, pH 7.5) using 25 μg of a membrane preparation from transiently transfected HEK293 cells. Ligand is added to membranes in the presence of 10 μM GDP and 0.1 nM [³⁵S]GTPγS (1200 Ci/mmol) and incubated at 30° C. for 30 min. Bound GTPγS is separated from unbound using the Brandel harvester (Gaithersburg, Md.) and counted with a liquid scintillation counter.

The S1P receptor modulators and agonists that are employed in the methods and kits of the present invention can be administered via oral, parenteral (such as subcutaneous, intraveneous, intramuscular, intrasternal and infusion techniques), rectal, intranasal or topical routes.

The S1P receptor modulators and agonists that are employed in the methods and kits of the present invention compounds may be administered alone or in combination with pharmaceutically acceptable carriers or diluents by either of the routes previously indicated, and such administration may be carried out in single or multiple doses. They can be administered in a wide variety of different dosage forms, i.e., they may be combined with various pharmaceutically acceptable inert carriers in the form of tablets, capsules, lozenges, troches, hard candies, powders, sprays, creams, salves, suppositories, jellies, gels, pastes, lotions, ointments, aqueous suspensions, injectable solutions, elixirs, syrups, and the like. Such carriers include solid diluents or fillers, sterile aqueous media and various non-toxic organic solvents, etc. Moreover, oral pharmaceutical compositions can be suitably sweetened and/or flavored.

For oral administration, tablets containing various excipients such as microcrystalline cellulose, sodium citrate, calcium carbonate, dicalcium phosphate and glycine may be employed along with various disintegrants such as starch (and preferably corn, potato or tapioca starch), alginic acid and certain complex silicates, together with granulation binders like polyvinylpyrrolidone, sucrose, gelatin and acacia. Additionally, lubricating agents such as magnesium stearate, sodium lauryl sulfate and talc are often very useful for tabletting purposes. Solid compositions of a similar type may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions of a compound of the present invention in either sesame or peanut oil or in aqueous propylene glycol may be employed. The aqueous solutions should be suitably buffered (preferably pH greater than 8) if necessary and the liquid diluent first rendered isotonic. These aqueous solutions are suitable for intravenous injection purposes. The oily solutions are suitable for intra-articular, intra-muscular and subcutaneous injection purposes. The preparation of all these solutions under sterile conditions is readily accomplished by standard pharmaceutical techniques well known to those skilled in the art.

1. A method of treating an autoimmune disease in a mammalian patient, preferably a human, in need of such treatment, comprising administering to the patient a compound that is an S1P receptor modulator or agonist, or pharmaceutically acceptable salt or prodrug thereof, in accordance with a dosing regimen that is determined with reference to the blood lymphocyte count of the patient.

2. A method according to claim 1, wherein the autoimmune disease is multiple sclerosis.

3. A method according to claim 1, wherein the daily dosage of the S1P receptor modulator or agonist is increased if the patient's blood lymphocyte count is greater than the Blood Lymphocyte Target Level and decreased if the patient's blood lymphocyte count is lower than the Blood Lymphocyte Target Level.

4. A method according to claim 3, wherein the daily dosage that is determined with reference to the blood lymphocyte count of the patient is adjusted, if necessary, so that it is not less than about 0.5 mg and not greater than about 5.0 mg.

5. A method according to claim 4, wherein the daily dosage that is determined with reference to the blood lymphocyte count of the patient is adjusted, if necessary, so that it is not less than about 0.2 mg and not greater than about 5.0 mg.

6. A method according to claim 4, wherein the daily dosage that is determined with reference to the blood lymphocyte count of the patient is adjusted, if necessary, so that it is not less than about 0.25 mg and not greater than about 5.0 mg.

7. A method according to claim 3, comprising:
(a) administering the S1P receptor modulator or agonist, or a pharmaceutically acceptable salt or prodrug thereof, to the patient at an Introductory Dosage that is between about 1.0 mg/day and about 2.0 mg/day, for an Introductory Period;
(b) comparing the blood lymphocyte count of the patient determined at the end of the Introductory Period with the Blood Lymphocyte Target Level;
(c) (i) if the blood lymphocyte count of the patient determined at the end of the Introductory Period is equal to or within the range of the Blood Lymphocyte Target Level, administering the S1P receptor modulator or agonist to the subject at a Maintenance Dosage that is about the same as the Introductory Dosage; or,
(ii) if the blood lymphocyte count of the patient determined at the end of the Introductory Period is lower than the Blood Lymphocyte Target Level, administering the S1P receptor modulator or agonist to the subject for a Post-Introductory Period at a Post-Introductory Dosage that is the greater of (A) between about 40% and about 60% less than the Introductory Dosage and (B) about 0.5 mg/day; or,
(iii) if the blood lymphocyte count of the patient determined at the end of the Introductory Period is greater than the Blood Lymphocyte Target Level, administering the S1P receptor modulator or agonist to the subject for a Post-Introductory Period at a Post-Introductory Dosage that is the lesser of (A) between about 90% and about 110% greater than the Introductory Dosage and (B) about 5.0 mg/day;
(d) if, during the Post-Introductory Period, the patient was administered the S1P receptor modulator or agonist, or a pharmaceutically acceptable salt or prodrug thereof, at a Post-Introductory Dosage that was determined in accordance with step (c) (ii) or step (c) (iii) above, then, comparing the blood lymphocyte count of the patient determined at the end of the Post-Introductory Period with the Blood Lymphocyte Target Level;
(e) (i) if the blood lymphocyte count of the patient determined at the end of the Post-Introductory Period is equal to or within the range of the Blood Lymphocyte Target Level, administering the S1P receptor modulator or agonist to the patient at a Maintenance Dosage that is about the same as the Post-Introductory Dosage; or,
(ii) if the blood lymphocyte count of the patient determined at the end of the Post-Introductory Period is lower than the Blood Lymphocyte Target Level, administering the S1P receptor modulator or agonist to the subject at a Maintenance Dosage that is the greater of (A) between about 40% and about 60% less than the Post-Introductory Dosage and (B) about 0.5 mg/day; or,
(iii) if the blood lymphocyte count of the patient determined at the end of the Post-Introductory Period is greater than the Blood Lymphocyte Target Level, administering the S1P receptor modulator or agonist to the subject at a Maintenance Dosage that is the lesser of (A) between about 90% and about 110% greater than the Post-Introductory Dosage and (B) about 5.0 mg/day.

8. A method according to claim 7, wherein the daily dosage set forth in steps (c)(ii)(B) and (e)(ii)(B) is 0.2 mg/day rather than 0.5 mg/day.

9. A method according to claim 7, wherein the daily dosage set forth in steps (c)(ii)(B) and (e)(ii)(B) is 0.25 mg/day rather than 0.5 mg/day.

10. A method according to claim 7, wherein the Introductory Dosage is about 1.0 mg/day.

11. A method according to claim 7, wherein the Introductory Period is about 14 days.

12. A method according to claim 7, wherein the Post-Introductory Period is about 14 days.

13. A method according to claim 7, wherein the Blood Lymphocyte Target Level is a blood lymphocyte count that is greater than about 0.2×10e9/L and less than about 0.5×10e9/L.

14. A method according to claim 7, wherein the Blood Lymphocyte Target Level is a blood lymphocyte count that is greater than about 0.3×10e9/L and less than about 0.7×10e9/L.

15. A method according to claim 7, wherein, if the blood lymphocyte count referred to in step “b” is lower than the Blood Lymphocyte Target Level, the S1P receptor modulator or agonist is administered to the subject during the Post-Introductory Period, in accordance with step (c) (ii), at a dosage that is about 50% less than the Introductory Dosage.

16. A method according to claim 7, wherein, if the blood lymphocyte count referred to in step “b” is greater than the Blood Lymphocyte Target Level, the S1P receptor modulator or agonist is administered to the subject during the Post-Introductory Period, in accordance with step (c) (iii), at a dosage that is about 100% greater than the Introductory Dosage.

17. A method according to claim 7, wherein the Blood Lymphocyte Target Level to which the patient's blood lymphocyte count is compared at the end of the Introductory Period, as specified in step “c”, is different from the Blood Lymphocyte Target Level to which the patient's blood lymphocyte count is compared at the end of the Post-Introductory Period, as specified in step “e”.

18. A method according to claim 7, wherein the Blood Lymphocyte Target Level to which the patient's blood lymphocyte count is compared at the end of the Introductory Period, as specified in step “c”, is a blood lymphocyte count that is greater than 0.2×10e9/L and less than 0.5×10e9/L, and the Blood Lymphocyte Target Level to which the patient's blood lymphocyte count is compared at the end of the Post-Introductory Period, as specified in step “e”, is a blood lymphocyte count that is greater than 0.2×10e9/L and less than 1.0×10e9/L.

19. A method according to claim 1, wherein the S1P receptor modulator or agonist is a compound of Formula Ia or Ib 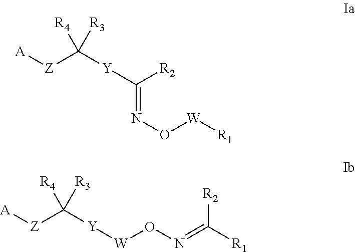
in which:
A is chosen from —C(O)OR₅, —OP(O)(OR₅)₂, —P(O)(OR₅)₂, —S(O)₂OR₅, —P(O)(R₅)OR₅ and 1H-tetrazol-5-yl; wherein each R₅ is independently chosen from hydrogen and C₁₋₆alkyl;
W is chosen from a bond, C₁₋₃alkylene, C₂₋₃alkenylene;
Y is chosen from C₆₋₁₀aryl and C₂₋₉heteroaryl; wherein any aryl or heteroaryl of Y can be optionally substituted with 1 to 3 radicals chosen from halo, hydroxy, nitro, C₁₋₆alkyl, C₁₋₆alkoxy, halo-substituted C₁₋₆alkyl and halo-substituted C₁₋₆alkoxy;
Z is chosen from: 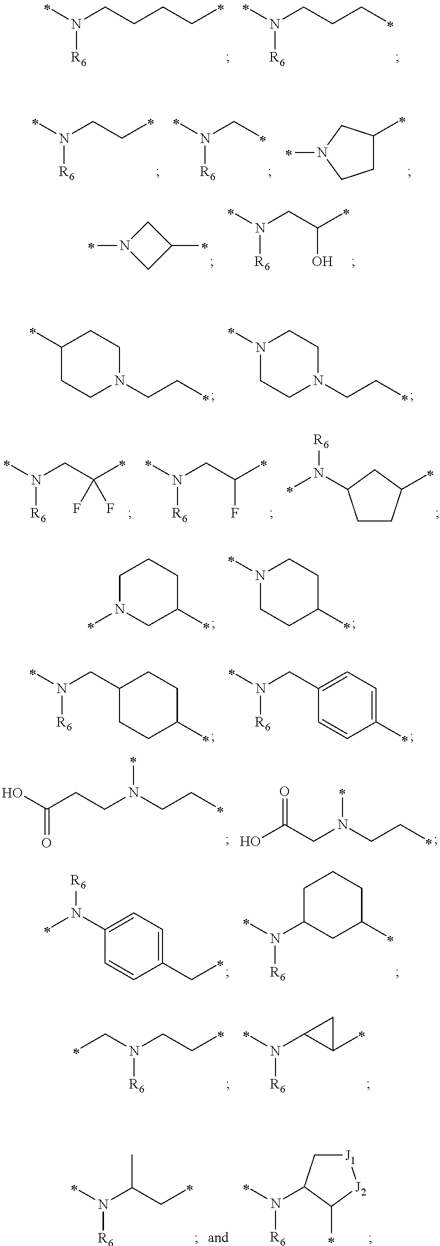
wherein the left and right asterisks of Z indicate the point of attachment between —C(R₃)(R₄)— and A of Formula Ia or Ib, respectively; R₆ is chosen from hydrogen and C₁₋₆alkyl; and J₁ and J₂ are independently methylene or a heteroatom chosen from S, O and NR₅; wherein R₅ is chosen from hydrogen and C₁₋₆alkyl; and any alkylene of Z can be further substituted by one to three radicals chosen from halo, hydroxy, C₁₋₆alkyl; or R₆ can be attached to a carbon atom of Y to form a 5-7 member ring;
R₁ is chosen from C₆₋₁₀aryl and C₂₋₉heteroaryl; wherein any aryl or heteroaryl of R₁ is optionally substituted by a radical chosen from C₆₋₁₀arylC₀₋₄alkyl, C₂₋₉heteroarylC₀₋₄alkyl, C₃₋₈cycloalkylC₀₋₄alkyl, C₃₋₅heterocycloalkylC₀₋₄alkyl or C₁₋₆alkyl; wherein any aryl, heteroaryl, cycloalkyl or heterocycloalkyl group of R₁ can be optionally substituted by one to five radicals chosen from halo, C₁₋₆alkyl, C₁₋₆alkoxy, halo-substituted-C₁₋₆alkyl and halo-substituted-C₁₋₆alkoxy; and any alkyl group of R₁ can optionally have a methylene replaced by an atom or group chosen from —S—, —S(O)—, —S(O)₂—, —NR₅— and —O—; wherein R₅ is chosen from hydrogen or C₁₋₆alkyl;
R₂ is chosen from hydrogen, C₁₋₆alkyl, C₂₋₆alkenyl, C₂₋₆alkynyl and halo substituted C₁₋₆alkyl;
R₃ and R₄ are independently chosen from hydrogen, C₁₋₆alkyl, halo, hydroxy, C₁₋₆alkoxy, halo-substituted C₁₋₆alkyl and halo-substituted C₁₋₆alkoxy; and the N-oxide derivatives, prodrug derivatives, protected derivatives, individual isomers and mixtures of isomers thereof; and the pharmaceutically acceptable salts and solvates of such compounds.

20. A method according to claim 1, wherein the S1P receptor modulator or agonist is 1-{4-[1-(4-cyclohexyl-3-trifluoromethyl-benzyloxyimino)-ethyl]-2-ethyl-benzyl}-azetidine-3-carboxylic acid, or a pharmaceutically acceptable salt or prodrug thereof.

21. A method according to claim 7, wherein the S1P receptor modulator or agonist is the hemifumarate salt of 1-{4-[1-(4-cyclohexyl-3-trifluoromethyl-benzyloxyimino)-ethyl]-2-ethyl-benzyl}-azetidine-3-carboxylic acid.

22. A kit containing daily units of medication of an S1P receptor modulator or agonist, or a pharmaceutically acceptable salt or prodrug thereof, for the treatment of multiple sclerosis in a mammalian patient, preferably a human, in need of such treatment, wherein the daily dosage of the dosage of the S1P receptor modulator or agonist, or a pharmaceutically acceptable salt or prodrug thereof, was determined with reference to the blood lymphocyte count of the patient.

23. A kit according to claim 22, wherein the daily dosage of the S1P receptor modulator or agonist, or a pharmaceutically acceptable salt or prodrug thereof, is the Maintenance Dosage that was determined using the following method:
(a) administering the S1P receptor modulator or agonist, or a pharmaceutically acceptable salt or prodrug thereof, to the patient at an Introductory Dosage that is between about 1.0 mg/day and about 2.0 mg/day, for an Introductory Period;
(b) comparing the blood lymphocyte count of the patient determined at the end of the Introductory Period with the Blood Lymphocyte Target Level;
(c) (i) if the blood lymphocyte count of the patient determined at the end of the Introductory Period is equal to or within the range of the Blood Lymphocyte Target Level, administering the S1P receptor modulator or agonist to the subject at a Maintenance Dosage that is about the same as the Introductory Dosage; or,
(ii) if the blood lymphocyte count of the patient determined at the end of the Introductory Period is lower than the Blood Lymphocyte Target Level, administering the S1P receptor modulator or agonist to the subject for a Post-Introductory Period at a Post-Introductory Dosage that is the greater of (A) between about 40% and about 60% less than the Introductory Dosage and (B) about 0.5 mg/day; or,
(iii) if the blood lymphocyte count of the patient determined at the end of the Introductory Period is greater than the Blood Lymphocyte Target Level, administering the S1P receptor modulator or agonist to the subject for a Post-Introductory Period at a Post-Introductory Dosage that is the lesser of (i) between about 90% and about 110% greater than the Introductory Dosage and (ii) about 5.0 mg/day;
(d) if, during the Post-Introductory Period, the patient was administered the S1P receptor modulator or agonist, or a pharmaceutically acceptable salt or prodrug thereof, at a Post-Introductory Dosage that was determined in accordance with step (c) (ii) or step (c) (iii) above, then, comparing the blood lymphocyte count of the patient determined at the end of the Post-Introductory Period with the Blood Lymphocyte Target Level;
(e) (i) if the blood lymphocyte count of the patient determined at the end of the Post-Introductory Period is equal to or within the range of the Blood Lymphocyte Target Level, administering the S1P receptor modulator or agonist to the patient at a Maintenance Dosage that is about the same as the Post-Introductory Dosage; or,
(ii) if the blood lymphocyte count of the patient determined at the end of the Post-Introductory Period is lower than the Blood Lymphocyte Target Level, administering the S1P receptor modulator or agonist to the subject at a Maintenance Dosage that is the greater of (A) between about 40% and about 60% less than the Post-Introductory Dosage and (B) about 0.5 mg/day; or,
(iii) if the blood lymphocyte count of the patient determined at the end of the Post-Introductory Period is greater than the Blood Lymphocyte Target Level, administering the S1P receptor modulator or agonist to the subject at a Maintenance Dosage that is the lesser of (A) between about 90% and about 110% greater than the Post-Introductory Dosage and (B) about 5.0 mg/day.

24. A kit according to claim 22, wherein the S1P receptor modulator or agonist is 1-{4[1-(4-cyclohexyl-3-trifluoromethyl-benzyloxyimino)-ethyl]-2-ethyl-benzyl}-azetidine-3-carboxylic acid, or a pharmaceutically acceptable salt or prodrug thereof.

25. A kit according to claim 22, wherein the S1P receptor modulator or agonist is the hemifumarate salt of 1-{4-[1-(4-cyclohexyl-3-trifluoromethyl-benzyloxyimino)-ethyl]-2-ethyl-benzyl}-azetidine-3-carboxylic acid.

26. A kit according to claim 22, wherein the Blood Lymphocyte Target Level to which the patient's blood lymphocyte count is compared at the end of the Introductory Period, as specified in step “c”, is a blood lymphocyte count that is greater than 0.2×10e9/L and less than 0.5×10e9/L, and the Blood Lymphocyte Target Level to which the patient's blood lymphocyte count is compared at the end of the Post-Introductory Period, as specified in step “e”, is a blood lymphocyte count that is greater than 0.2×10e9/L and less than 1.0×10e9/L.

27. A method according to claim 7, wherein the Introductory Dosage is about 2.0 mg/day.

28. A method according to claim 7, wherein the Introductory Period is about 7 days.

29. A method according to claim 7, wherein the Post-Introductory Period is about 7 days.

30. A method according to claim 7, wherein the length of the Introductory Period and the Post-Introductory Period are, independently, from about 7 to about 14 days.

31. A method according to claim 7, wherein the Blood Lymphocyte Target Level is a blood lymphocyte count that is greater than about 0.2×10e9/L and less than about 1.0×10e9/L.

32. A method according to claim 7, wherein the Blood Lymphocyte Target Level is a blood lymphocyte count that is greater than about 0.3×10e9/L and less than about 1.0×10e9/L.

33. A method according to claim 7, wherein the Blood Lymphocyte Target Level is a blood lymphocyte count that is greater than about 0.2×10e9/L and less than about 0.7×10e9/L.

34. A method according to claim 7, wherein the Blood Lymphocyte Target Level used in step (c) and the Blood Lymphocyte Target Level used in step (e) are, independently, within one of the following ranges:
(A) greater than about 0.2×10e9/L and less than about 1.0×10e9/L;
(B) greater than about 0.2×10e9/L and less than about 0.5×10e9/L;
(C) greater than about 0.2×10e9/L and less than about 0.7×10e9/L;
(D) greater than about 0.3×10e9/L and less than about 0.7×10e9/L; and
(E) greater than about 0.3×10e9/L and less than about 1.0×10e9/L.
